(19) United States
(12) Patent Application Publication
(10) Pub. No.: US 2023/0302027 A1
(43) Pub. Date: Sep. 28, 2023

(54) HOMOGENEOUS HASHISH PRODUCT

(71) Applicant: HEXO OPERATIONS INC., GATINEAU (CA)

(72) Inventors: Renato Devien Durbano, Gatineau (CA); Todd Neault, Gatineau (CA)

(21) Appl. No.: 18/024,137

(22) PCT Filed: Sep. 2, 2021
(86) PCT No.: PCT/CA2021/051222

§ 371 Date: Mar. 1, 2023

Related U.S. Application Data
(60) Provisional application No. 63/073,549, filed on Sep. 2, 2020.

(51) Int. Cl. A61K 31/00; A61K 36/185

(52) U.S. Cl. CPC A61K 31/658; A61K 36/185

(57) ABSTRACT

Traditional methods of hash production are very labor intensive, and do not yield a product that is homogeneous, whether within a unit or across multiple units from a batch. The present disclosure relates to a hash product comprising a cohesive mass of isolated cannabis trichomes and a marker, where the distribution of the marker is substantially homogeneous throughout the product. For example, the detectable marker can be distributed in at least 80 vol.% of the hashish product. Additionally, or alternatively, the hashish product includes a first content of the marker in a core portion thereof and a second content of the marker in a peripheral portion thereof, where the first content and the second content are present in a ratio first content / second content of from 0.85 to 1.15. Such hash product may be consumed by inhalation or ingestion.

### CROSS-REFERENCE TO RELATED APPLICATION

The present application claims the benefit of U.S. Provisional Pat. Application Serial No. 63/073,549 filed on Sep. 2, 2020 by Durbano et al. The contents of the above-referenced document are incorporated herein by reference in their entirety.

### TECHNICAL FIELD

This application generally relates to the field of methods of manufacturing cannabis-based consumer products and, more specifically, to methods of manufacturing hashish products at an industrial scale.

### BACKGROUND

With stage-wise legalization of cannabis-based consumer products in Canada and eventually in various other areas in the world, advancements in extraction technology, industrial scale production and accessibility to a wide variety of forms have accelerated to fulfill emerging demands.

Hashish (or hash) is a concentrated derivative of the dried resin glands, known as trichomes, of mature and unpollinated female cannabis plants. Hash contains the same active ingredients as marijuana - including cannabinoids such as tetrahydrocannabinol and others -although at higher concentrations than the un-sifted buds or leaves from which dried marijuana is made, which is tantamount to higher potency. The trichomes may be removed from the plant material by mechanical or chemical means.

Chemical separation methods generally use an organic solvent such as ethanol, butane, or hexane to dissolve the trichomes; the solvent is then evaporated or boiled off (purged) to yield a resin, called honey oil or “hash oil”. However, due to concerns of residual organic solvents in the hashish product, market demand has caused a shift to products produced using alternative separation methods.

Mechanical separation may be used to remove trichomes from the plant, such as sieving through a screen by hand or in motorized tumblers (called “dry-sift”), as described for example in WO 2019/161509. Another approach is to submerge the cannabis plants in icy water and agitate to separate the trichomes from the plant. Methods for separating trichomes from the cannabis plant are well-known in the art.

Separated trichomes have a powder appearance (referred to as “kief”) and are typically heated to have their moisture content fully removed. The resulting kief is subsequently pressed to obtain blocks of hash, the color and pliability of which can vary widely based on the source material, the extraction method, and the production conditions. For example, dry-sift pressed hashish is usually solid, whereas water-purified hashish - often called bubble hashish - is often a paste-like substance with varying hardness and pliability. The color of a hashish product is most commonly light to dark brown, but can also vary from transparent to yellow, tan, black, or red.

Hand or mechanical presses are often used to produce hash products. However, hand presses are too small and inefficient for commercial volume production, while mechanical presses may also be used, variability of the finished hash product result in an inconsistent product batch-over-batch. Furthermore, obtaining the desirable pliability and hardness requires a significant amount of “art” that is hardly reproduceable and the skills of the individual play a key role in defining the quality of the finished product - characteristics that are undesirable when designing and implementing industrial scale procedures.

Additionally, current methods of producing hash cannot ensure thorough mixing of the hashish components, and thus cannot ensure uniform and homogeneous distribution of hashish components. This leads to a hashish product with uneven and unpredictable distribution of cannabinoids throughout a unit of product, or across batches of product, which leads to inconsistent dosage and/or user experience. Additionally, difficulties in thoroughly mixing hashish components can lead to a lack of uniform texture, consistency, and color in the hashish product, which can be off-putting to a user and can signal other inconsistencies in the product. This challenge in consistently and homogeneously distributing components within a product unit and across batches have limited the development of hash within the legal cannabis industry. For example, mixing of kief from different cannabis strains or the addition of other components to a hashish product has not been widely explored due to inconsistent blending and resulting variability in dosage and/or user experience.

Considering the above, it would be highly desirable to be provided with a system or method that would at least partially alleviate the disadvantages of the existing technologies and afford a hash product having improved characteristics.

### SUMMARY

This Summary is provided to introduce a selection of concepts in a simplified form that are further described below in the Detailed Description. This Summary is not intended to identify key aspects or essential aspects of the claimed subject matter.

Broadly stated, in some embodiments, the present disclosure relates to a hashish product, comprising a cohesive mass of isolated cannabis trichomes and a detectable marker. The marker is substantially homogeneously distributed throughout the hashish product.

Broadly stated, in some embodiments, the present disclosure relates to a hashish product, comprising a cohesive mass of isolated cannabis trichomes and a detectable marker. The marker is distributed in at least 80%, or in at least 85%, or in at least 90 vol.%, or in at least 95 vol.%, or in at least 99%, or in 100% of the hashish product.

Broadly stated, in some embodiments, the present disclosure relates to a hashish product, comprising a cohesive mass of isolated cannabis trichomes and a detectable marker. The hashish product includes a first detectable content of the marker in a core portion thereof and a second detectable content of the marker in a peripheral portion thereof. The first content and the second content are present in a ratio first content / second content of from 0.85 to 1.15.

Broadly stated, in some embodiments, the present disclosure relates to a batch of hashish products, each hashish product in the batch of hashish products comprising a cohesive mass of isolated cannabis trichomes and a detectable marker, wherein the detectable marker in a discreet portion of a first hashish product is a first level of the marker, wherein the first level of the marker is within 15% of a second level of the marker, and wherein the second level is an average level of the marker in the batch of hashish products.

In some embodiments, the hashish product of the present disclosure may include one or more of the following features, in any combination:
a first level of the marker in a discreet portion of the product is within 15% of a second level of the marker, where the second level is an average marker level of the hashish product.
the ratio first content / second content is of from 0.85 to 1.10, or from 0.85 to 1.05, or from 0.85 to 1.00, or from 0.90 to 1.15, or from 0.90 to 1.10, or from 0.90 to 1.05, or from 0.90 to 1.00, or from 0.95 to 1.15, or from 0.95 to 1.10, or from 0.95 to 1.05, or from 0.95 to 1.00, or from 1.00 to 1.15, or from 1.00 to 1.15, or from 1.00 to 1.10, or from 1.00 to 1.05, or any value within any of these ranges.
the marker includes an endogenous component to the cannabis trichomes.
the marker includes an exogenous component to the cannabis trichomes.
the marker is a cannabinoid, a terpene, a flavonoid, chlorophyll, water, or any combination thereof.
the cohesive mass of isolated cannabis trichomes is made with kief.
the isolated cannabis trichomes are from one or more strain(s) of cannabis plant.
one or more additional components are incorporated into the hashish product.
the one or more additional components comprise one or more cannabinoid(s), one or more terpene(s), one or more flavonoid(s), water, one or more flavoring agent(s), one or more coloring agent(s), or any combinations thereof.
the one or more additional components comprise one or more cannabinoid(s), which is (are) provided in the form of a crude extract, a winterized extract, a distillate, an isolate, or any combinations thereof.
the hashish product comprises at least one cannabinoid.
the at least one cannabinoid is selected from the group consisting of tetrahydrocannabinol (THC), cannabidiol (CBD), cannabinol (CBN), and any combinations thereof.
the hashish product comprises a cannabinoid content of from about 5 wt.% to about 90 wt.%.
the cannabinoid content is up to about 60 wt.%, or up to about 50 wt.%, or up to about 40 wt.%, or up to about 30 wt.%.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF THE DRAWINGS

A detailed description of specific exemplary embodiments is provided herein below with reference to the accompanying drawings in which:

FIGS. 1A and 1B show a non-limiting flowchart example of a process for making a hashish product in accordance with an embodiment of the present disclosure.

FIG. 2 illustrates a non-limiting system implementing the method of FIG. 1A for manufacturing the hashish product.

FIG. 3 shows a non-limiting schematic representation of a distribution test wherein samples are taken from a hashish block.

In the drawings, exemplary embodiments are illustrated by way of example. It is to be expressly understood that the description and drawings are only for the purpose of illustrating certain embodiments and are an aid for understanding. They are not intended to be a definition of the limits of the invention.

### DETAILED DESCRIPTION

A detailed description of one or more embodiments of the invention is provided below along with accompanying figures that illustrate principles of the invention. The invention is described in connection with such embodiments, but the invention is not limited to any embodiment. The scope of the invention is limited only by the claims. Numerous specific details are set forth in the following description to provide a thorough understanding of the invention. These details are provided for the purpose of non-limiting examples and the invention may be practiced according to the claims without some or all these specific details. For sake of clarity, technical material that is known in the technical fields related to the invention has not been described in detail so that the invention is not unnecessarily obscured.

The present inventors have developed a hashish product and method of manufacturing same that addresses at least some of the above-identified problems.

The hashish product of the present disclosure has the form of a cohesive mass of isolated cannabis trichomes and includes a detectable marker where the marker is distributed substantially homogeneously in the product.

Without being bound by any theory, it is believed that the herein described homogeneity characteristics may allow, for example, improvement in the textural consistency, pliability and/or crumbliness of the hashish product. This in turn, may reduce / minimize quality control failures during large-scale manufacturing of the hashish product (e.g., quality control based on textural consistency, pliability and/or crumbliness). It is also believed that such hashish product may afford an enhanced and more consistent user experience in that the reduced crumbliness may lead to better segmentation during use of the hashish product thereby resulting in reduction of waste material during use. Further, the method of manufacture described herein may result in substantially fewer quality failures (e.g., based on textural consistency, pliability and/or crumbliness) and/or reduce waste materials during manufacturing of the hashish product, which is advantageous in the context of large-scale industrial production. The reduction of waste materials during manufacturing can be afforded with the process described herein in that this process allows the use of various strains of kief, which leads to less wasted materials that would require disposal thereof in other circumstances where one cannot obtain a homogeneous mixture of the various strains of kief. Further, hashish products with increased homogeneity deliver consistent amounts of cannabinoids, terpenes, flavonoids, and the like to the user during each use, thus providing a more consistently reproducible dosage and/or user experience.

Additionally, or alternatively, controlling homogeneity of the cohesive mass of isolated cannabis trichomes and other (optional) components included therein may provide hashish products that contain substantially homogeneous distribution of the herein described markers within single product units, and/or over multiple product units, and/or over multiple batches of product units. This in turn can be advantageous in view of increasing consumer demands for predictable dosage and/or user experience.

In some embodiments, the hashish product of the present disclosure and the process for making same described herein afford several advantageous characteristics to the hashish product that will become apparent to the person of skill in view of the present disclosure.

### Hashish Product

The hashish product of the present disclosure has the form of a cohesive mass of isolated cannabis trichomes and includes a detectable marker where the marker is distributed substantially homogeneously in the product.

By “distributed substantially homogeneously” or “substantially homogeneous distribution”, it is meant that the proportion of detectable marker is uniform throughout the hashish product as discussed elsewhere in this text.

In the context of the present disclosure, the marker will be substantially homogeneously distributed when a first marker level in a discreet portion (also called “sample”) of the hashish product is within 15% of a second marker level. In some embodiments, the second marker level can be the average marker level detected in the hashish product. The average marker level may be determined as an average from quantification of a single hashish unit or over the entire batch of a hashish product, or the average marker level may be a value set during production. In some embodiments, the first marker level is determined in a first portion (e.g., a core portion) of the hashish product and the second marker level is determined in a second portion (e.g., a peripheral portion) of the hashish product, as measured with the marker distribution test described elsewhere in this text. As would be understood by a person of skill in the art, the first and second marker levels can be determined in any two different portions of the hashish product. The marker may be substantially homogeneously distributed within a unit of hashish product, across multiple units of hashish product, or across all hashish product units produced within a batch.

In some embodiments, the first level of the marker may be within 15%, 14%, 13%, 12%, 11%, 10%, 9%, 8%, 7%, 6%, 5%, 4%, 3%, 2%, or 1% of the second level of the marker. In one specific example, the first level is within 10% of the second level. In other words, the standard deviation and/or the variance may be within 10%.

In some embodiments, the detection and measurement of the marker may be done during the manufacturing of the hashish product using suitable equipment / procedures, such as off-line, in-line, on-line, or at-line equipment / procedures. On-line and in-line analyses differ essentially from the off-line and at-line analyses in that the time in which information about process or material properties is obtained is shorter than the time in which these properties change. This means that on-line and in-line analyses permit continuous process control typically using sensor-based equipment / procedures. Off-line and at-line analyses, on the other hand, are characterized by manual sampling followed by discontinuous sample preparation, measurement and evaluation typically using laboratory-based equipment / procedures. For example, for in-line analysis, a sensor can be placed in a process vessel or stream of flowing material to conduct the analysis; for on-line analysis, a sensor can be connected to the process, and conduct automatic sampling.

For example, the marker can be detected in at least 80 vol.%, or in at least 85 vol.%, or in at least 90 vol.%, or in at least 95 vol.%, or in at least 99 vol.%, or in 100 vol.% of the hashish product depending on specific implementations of the present disclosure.

Alternatively, or additionally, the levels (or contents) of the detectable marker in the hashish product of the present disclosure is substantially homogeneous, such that the hashish product includes a first marker content in a first portion thereof and a second marker content in a second portion thereof, where the first marker content and the second marker content are substantially identical. For example, the first marker content and the second marker content are present in a ratio first / second markers of from 0.85 to 1.15, from 0.85 to 1.10, or from 0.85 to 1.05, or from 0.85 to 1.00, or from 0.90 to 1.15, or from 0.90 to 1.10, or from 0.90 to 1.05, or from 0.90 to 1.00, or from 0.95 to 1.15, or from 0.95 to 1.10, or from 0.95 to 1.05, or from 0.95 to 1.00, or from 1.00 to 1.15, or from 1.00 to 1.15, or from 1.00 to 1.10, or from 1.00 to 1.05, or any value within any of these ranges, such as for example at least 0.90, at least 0.95, 1.00, 1.05 or less, 1.10 or less or 1.15 or less. For example, the first portion can be a core portion and the second portion can be a peripheral portion, where the marker content and the ratio of first / second markers can be determined based on the marker distribution test described later in this text.

As used herein, the term “cannabis trichomes” or “trichomes” generally refers to crystal-shaped outgrowths or appendages (also called resin glands) on cannabis plants typically covering the leaves and buds. Trichomes produce hundreds of known cannabinoids, terpenes, and flavonoids that make cannabis strains potent, unique, and effective.

As used herein, the term “cannabis plant(s)”, encompasses wild type Cannabis (including but not limited to the species species Cannabis sativa, Cannabis indica and Cannabis ruderalis) and also variants thereof, including cannabis chemovars (or “strains”) that naturally contain different amounts of the individual cannabinoids.

As used herein, the term “isolated cannabis trichomes” refers to trichomes that have been separated from cannabis plant material plant using any method known in the art. For example, and without wishing to be limiting in any manner, the isolated cannabis trichomes may be obtained by a chemical separation method using a solvent such as ethanol, butane or hexane to dissolve the lipophilic desirable resin; the solvent is then purged to produce the desirable resin (“honey oil” or “hash oil”). Such methods are known in the art, though the potential for residual organic solvents in the hashish product is often undesirable to consumers.

Other methods for obtaining isolated cannabis trichomes include, but are not limited to solventless extraction methods, including but not limited to mechanical separation of trichomes from the plant, such as by sieving through a screen by hand or in motorized tumblers (see for example WO 2019/161509), or by submerging the cannabis plants in icy water (see for example US2020/0261824, which is herein incorporated by reference) and agitating to separate the trichomes from the plant and drying the trichomes. The details of various methods for separating trichomes from the cannabis plant are well-known in the art. Isolated cannabis trichomes obtained by mechanical separation of trichomes from the cannabis plant material is typically referred to as “kief” (also “keef” or “kif”) and has a powdery appearance. The moisture content may be fully or partially removed, often using heat and the finished kief is subsequently pressed or formed to obtain a hashish product. Typically, some residual plant material remains in the finished kief and thus in the resulting hashish product. In preferred embodiments of the present disclosure, the isolated cannabis trichomes is in the form of kief.

The isolated cannabis trichomes forming the hashish product of the present disclosure may originate from one or more than one strain of cannabis plant. It is known amongst consumers of hashish and other cannabis products that using isolated cannabis trichomes produced from more than one strain of cannabis plant allows a user to tune the psychoactive and/or entourage effect obtained by consuming the product. The mixing of cannabis plant strains may also allow to adjust the final concentration of a component of the product, for example but not limited to the cannabinoid content. Additionally, use of more than one strain allows for improved product and waste management - important in commercial production.

The hashish product of the present disclosure also comprises a marker. As used herein, the term “marker” encompasses a detectable chemical entity in the hashish product. In some embodiments, the marker may serve as an indicator for the quality, and more specifically of the homogeneity, of the hashish product. The marker may be endogenous or exogenous to the isolated cannabis trichomes. A marker that is “endogenous” to the isolated cannabis trichomes means a chemical entity that is naturally present in the strain(s) of isolated cannabis trichomes used to produce the specific hashish product. An endogenous marker can therefore originate from the cannabis plant material used to produce the isolated cannabis trichome. The reader will appreciate that while a given marker may originate from the cannabis plant material used to produce the isolated cannabis trichomes, the same marker may additionally be physically added to the isolated cannabis trichomes so as to increase the contents thereof in the product, thereby facilitating its detectability. For the purpose of the present specification, the marker in such cases is still considered as an “endogenous” marker. A marker that is “exogenous” to the isolated cannabis trichomes means a chemical entity that is physically added as an “additional component” (defined elsewhere in this disclosure) to the isolated cannabis trichomes and that is not naturally present in the specific strain(s) of isolated cannabis trichomes used to produce the specific hashish product; the exogenous marker may originate from a cannabis plant or may originate from sources other than cannabis.

The marker in the hashish product of the present disclosure may be any suitable marker that is detectable using quantitative methods. For example, and without wishing to be limiting in any manner, the marker may be a component of the isolated cannabis trichomes that is detectable using any suitable technique, such as for example Gas Chromatography/ Mass Spectrometry (GC/MS), High Pressure Liquid Chromatography (HPLC), Gas Chromatography/ Flame Ionization Detection (GC/FID), infra-red spectrum (IR) spectroscopy, ultra-violet spectrum (UV) spectroscopy, Raman spectroscopy, and the like.

For example, the marker may be one or more of the following: cannabinoid, a terpene, a flavonoid, chlorophyll, water, or any combination thereof. As is known in the art, chlorophyll is a green photosynthetic pigment found in plants, algae, and cyanobacteria; its presence in the hashish product can be due to residual cannabis plant matter found in the product and/or may be added to the isolated cannabis trichomes in the form of an exogenous marker. Similarly, the water content of the hashish may be due to residual moisture in the kief or to the addition of water during the productions process.

As used herein, the term “cannabinoid” generally refers to any chemical compound that acts upon a cannabinoid receptor such as CB1 and CB2. A cannabinoid may include endocannabinoids (produced naturally by humans and animals), phytocannabinoids (found in cannabis and some other plants), and synthetic cannabinoids (manufactured artificially, for example cannabinoids produced in yeast, for example as described in WO WO2018/148848). Examples of suitable phytocannabinoids include, but are not limited to, cannabichromanon (CBCN), cannabichromene (CBC), cannabichromevarin (CBCV), cannabicitran (CBT), cannabicyclol (CBL), cannabicyclovarin (CBLV), cannabidiol (CBD), cannabidiol monomethylether (CBDM), cannabidiol-C4 (CBD-C4), cannabidiorcol (CBD-C1), cannabidiphorol (CBDP), cannabidivarin (CBDV), cannabielsoin (CBE), cannabifuran (CBF), cannabigerol (CBG), cannabigerol monomethylether (CBGM), cannabigerolic acid (CBGA), cannabigerovarin (CBGV), cannabinodiol (CBND), cannabinodivarin (CBVD), cannabinol (CBN), cannabinol methylether (CBNM), cannabinol propyl variant (CBNV), cannabinol-C2 (CBN-C2), cannabinol-C4 (CBN-C4), cannabiorcol (CBN-C1), cannabiripsol (CBR), cannabitriol (CBO), cannabitriolvarin (CBTV), cannabivarin (CBV), dehydrocannabifuran (DCBF), Δ7-cis-iso tetrahydrocannabivarin, tetrahydrocannabinol (THC), Δ9-tetrahydrocannabionolic acid B (THCA-B), Δ9-tetrahydrocannabiorcol (THC-C1), tetrahydrocannabivarinic acid (THCVA), tetrahydrocannabivarin (THCV), ethoxy-cannabitriolvarin (CBTVE), trihydroxy-Δ9-tetrahydrocannabinol (triOH-THC), 10-ethoxy-9hydroxy-Δ6a-tetrahydrocannabinol, 8,9-dihydroxy-Δ6a-tetrahydrocannabinol, 10-oxo-Δ6a-tetrahydrocannabionol (OTHC), 3,4,5,6-tetrahydro-7-hydroxy-α-α-2-trimethyl-9-n-propyl-2, 6-methano-2H-1-benzoxocin-5-methanol (OH-iso-HHCV), Δ6a,10a-tetrahydrocannabinol (Δ6a,10a-THC), Δ8-tetrahydrocannabivarin (Δ8-THCV), Δ9-tetrahydrocannabiphorol (Δ9-THCP), Δ9-tetrahydrocannabutol (Δ9-THCB), derivatives of any thereof, and combinations thereof. Further examples of suitable cannabinoids are discussed in at least WO2017/190249 and U.S. Pat. Application Pub. No. US2014/0271940, which are each incorporated by reference herein in their entirety.

Cannabidiol (CBD) means one or more of the following compounds: Δ2-cannabidiol, Δ5-cannabidiol (2-(6-isopropenyl-3-methyl-5-cyclohexen-l-yl)-5-pentyl-l,3-benzenediol); Δ4-cannabidiol (2-(6-isopropenyl-3-methyl-4-cyclohexen-l-yl)-5-pentyl-l,3-benzenediol); Δ3-cannabidiol (2-(6-isopropenyl-3-methyl-3-cyclohexen-l-yl)-5-pentyl-l,3-benzenediol); Δ3,7-cannabidiol (2-(6-isopropenyl-3-methylenecyclohex-l-yl)-5-pentyl-l,3-benzenediol); Δ2-cannabidiol (2-(6-isopropenyl-3-methyl-2-cyclohexen-l-yl)-5-pentyl-l,3-benzenediol); Δ1-cannabidiol (2-(6-isopropenyl-3-methyl-l-cyclohexen-l-yl)-5-pentyl-l,3-benzenediol); and Δ6-cannabidiol (2-(6-isopropenyl-3-methyl-6-cyclohexen-l-yl)-5-pentyl-l,3-benzenediol). In a preferred embodiment, and unless otherwise stated, CBD means Δ2-cannabidiol.

Tetrahydrocannabinol (THC) means one or more of the following compounds: Δ8-tetrahydrocannabinol (Δ8-THC), Δ9-cis-tetrahydrocannabinol (cis-THC), Δ9-tetrahydrocannabinol (Δ9-THC), Δ9-tetrahydrocannabinolic acid A (THCA-A), Δ10-tetrahydrocannabinol (Δ10-THC), Δ9-tetrahydrocannabinol-C4 (THC-C4), Δ9-tetrahydrocannabinolic acid-C4 (THCA-C4), synhexyl (n-hexyl-Δ3THC). In a preferred embodiment, and unless otherwise stated, THC means one or more of the following compounds: Δ9-tetrahydrocannabinol and Δ8-tetrahydrocannabinol.

Examples of suitable synthetic cannabinoids include, but are not limited to, naphthoylindoles, naphthylmethylindoles, naphthoylpyrroles, naphthylmethylindenes, phenylacetylindoles, cyclohexylphenols, tetramethylcyclopropylindoles, adamantoylindoles, indazole carboxamides, quinolinyl esters, and combinations thereof.

A cannabinoid may be in an acid form or a non-acid form, the latter also being referred to as the decarboxylated form since the non-acid form can be generated by decarboxylating the acid form. Within the context of the present disclosure, where reference is made to a specific cannabinoid, the cannabinoid can be in its acid, its non-acid form, or be a mixture of both acid and non-acid forms.

As used herein, the term “terpene” generally refers to a class of chemical components comprised of the fundamental building block of isoprene, which can be linked to form linear structures or rings. Terpenes may include hemiterpenes (single isoprenoid unit), monoterpenes (two units), sesquiterpenes (three units), diterpenes (four units), sesterterpenes (five units), triterpenes (six units), and so on. At least some terpenes are expected to interact with, and potentiate the activity of, cannabinoids. Any suitable terpene may be used in the hashish product of the present invention. For example, terpenes originating from cannabis plant may be used, including but not limited to aromadendrene, bergamottin, bergamotol, bisabolene, borneol, 4-3-carene, caryophyllene, cineole/eucalyptol, p-cymene, dihydroj asmone, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpineol, 4-terpineol, terpinolene, and derivatives thereof. Additional examples of terpenes include nerolidol, phytol, geraniol, alpha-bisabolol, thymol, genipin, astragaloside, asiaticoside, camphene, beta-amyrin, thujone, citronellol, 1,8-cineole, cycloartenol, and derivatives thereof. Further examples of terpenes are discussed in U.S. Pat. Application Pub. No. US2016/0250270, which is herein incorporated by reference in its entirety for all purposes.

The term “flavonoid” as used herein refers to a group of phytonutrients comprising a polyphenolic structure. Flavonoids are found in diverse types of plants and are responsible for a wide range of functions, including imparting pigment to petals, leaves, and fruit. Any suitable flavonoid may be used in the hashish product of the present invention. For example, flavonoids originating from cannabis plant may be used, including but not limited to: apigenin, cannflavin A, cannflavin B, cannflavin C, chrysoeril, cosmosiin, flavocannabiside, homoorientin, kaempferol, luteolin, myricetin, orientin, quercetin, vitexin, and isovitexin.

### Additional Components

In some embodiments, the hashish product of the present disclosure may further comprise one or more additional component. For example, the one or more additional component can be added during the hashish product production process.

In some embodiments, the one or more additional component may be added to alter the characteristics of the hashish product, such as cannabinoid content, potency, entourage effect, odor, color, consistency, texture, pliability, and the like.

In some embodiments, the one or more additional component may be incorporated throughout the hashish product, or the one or more additional components may be distributed on at least a portion of a surface of the hashish product, for example as a coating. In some embodiments, the one or more additional component may be substantially homogeneously distributed on the at least portion of the surface of the hashish product. By “substantially homogeneously distributed”, it is meant that the amount of the one or more additional component is uniform on the at least portion of the surface of the hashish product.

The one or more additional component may be any suitable food grade and/or non-toxic composition or component known in the art. As will be recognized by those of skill in the art, the toxicity of each type of additional component may be dependent on the method of consumption of the hashish product. For example, in applications where smoke / vapor produced by the combustion / vaporization of hashish product is to be inhaled, suitable additional components may include, but are not limited to one or more cannabinoid, one or more terpene (also referred to herein as a “terpene blend”), one or more flavonoid, or any combination thereof.

In applications where the hashish product is to be ingested (as in an edible product), suitable additional components may additionally include one or more flavouring agent, one or more colouring agent, water, or any combination of any noted additional components. Additional components may be added to alter the characteristics of the hashish product, such as cannabinoid content, potency, entourage effect, odor, color, consistency, texture, pliability, and the like. The additional components may be added during the process to produce the hashish product, and similarly to the marker, may be substantially homogeneously distributed throughout the hashish product.

The one or more additional component may be a cannabinoid. The cannabinoid may be extracted from any suitable source material including, but not limited to, cannabis or hemp plant material (e.g., flowers, seeds, and trichomes) or may be manufactured artificially (for example cannabinoids produced in yeast, as described in WO WO2018/148848). Cannabinoids can be extracted from a cannabis or hemp plant material according to any procedure known in the art. For example, and without wishing to be limiting, a “crude extract” containing a cannabinoid may be obtained by extraction from plant materials using for example aliphatic hydrocarbons (such as propane, butane), alcohols (such as ethanol), petroleum ether, naphtha, olive oil, carbon dioxide (including supercritical and subcritical CO₂), chloroform, or any combinations thereof. Optionally, the crude extract may then be “winterized”, that is, extracted with an organic solvent (such as ethanol) to remove lipids and waxes (to produce a “winterized extract”), as described for example in US 7,700,368, US 2004/0049059, and US 2008/0167483, which are each herein incorporated by reference in their entirety. Optionally, the method for obtaining the cannabinoid may further include purification steps such as a distillation step to further purify, isolate or crystallize one or more cannabinoids, which is referred to in the art and herein as a “distillate”; US20160346339, which is incorporated herein by reference, describes a process for extracting cannabinoids from cannabis plant material using solvent extraction followed by filtration, and evaporation of the solvent in a distiller to obtain a distillate. The distillate may be cut with one or more terpenes. The crude extract, the winterized extract or the distillate may be further purified, for example using chromatographic and other separation methods known in the art, to obtain an “isolate”. Cannabinoid extracts may also be obtained using solventless extraction methods; for example, cannabis plant material may be subjected to heat and pressure to extract a resinous sap (“rosin”) containing cannabinoids; methods for obtaining rosin are well-known in the art.

In some embodiments of the present disclosure, the additional component may be one of more cannabinoid(s) selected from the group consisting of THC, CBD, CBN, and any combinations thereof.

The one or more additional component may be a terpene or a terpene blend. As used herein, the term “terpene” generally refers to a class of chemical components comprised of the fundamental building block of isoprene, which can be linked to form linear structures or rings. Terpenes may include hemiterpenes (single isoprenoid unit), monoterpenes (two units), sesquiterpenes (three units), diterpenes (four units), sesterterpenes (five units), triterpenes (six units), and so on. At least some terpenes are expected to interact with, and potentiate the activity of, cannabinoids. Any suitable terpene may be used in the hashish product of the present invention. For example, terpenes originating from cannabis plant may be used, including but not limited to aromadendrene, bergamottin, bergamotol, bisabolene, borneol, 4-3-carene, caryophyllene, cineole/eucalyptol, p-cymene, dihydroj asmone, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpineol, 4-terpineol, terpinolene, and derivatives thereof. Additional examples of terpenes include nerolidol, phytol, geraniol, alpha-bisabolol, thymol, genipin, astragaloside, asiaticoside, camphene, beta-amyrin, thujone, citronellol, 1,8-cineole, cycloartenol, hashishene, and derivatives thereof. Further examples of terpenes are discussed in U.S. Pat. Application Pub. No. US2016/0250270, which is herein incorporated by reference in its entirety for all purposes. The hashish product of the present disclosure may contain one or more terpene(s). The one or more terpene(s) may originate from the hashish, from an additional component, or both. In some embodiments, the hashish product of the present disclosure may include the one or more terpene(s) in an amount (the “terpene content”) sufficient for the user to experience a desired entourage effect when consuming the product. For example, the one or more terpene(s) may include hashishene. Without wishing to be bound by theory, hashishene is believed to be a terpene produced by rearrangement of myrcene that may be found in hashish after mechanical processing, and that may be responsible for the typical desirable “hashish flavour”.

The one or more additional component may be a flavoring agent. Any suitable flavoring agent known in the art may be used. For example, a natural or a synthetic flavoring agent.

For example, and without wishing to be limiting, the flavoring agent may be selected from the group consisting of extracts of cinnamon, monk fruit, cucumber, mint, orange, lime, citrus, cookie dough, chocolate, vanilla, jasmine, lychee, almond, banana, grape, pear, pineapple, pine, oak, apple, pumpkin, grapefruit, watermelon, cotton sugar, durian, longan, taro, sapote, toffee nut, caramel, lotus, mango, mangosteen, coconut, coffee, strawberry, passion fruit, blueberry, raspberry, kiwi, walnut, cocoa, cherimoya, custard apple, papaya, fig, plum, nectarine, peaches, guava, honeydew, jackfruit, kumquat, loquat, palm, pomelo, persimmon, quince, and tamarind, or any combinations thereof.

Other examples of suitable flavoring agents include, but are not limited to, mint oils, wintergreen, clove bud oil, cassia, sage, parsley oil, marjoram, lemon, orange, propenyl guaethol, heliotropine, 4-cis-heptenal, diacetyl, methyl-p-tert-butyl phenyl acetate, methyl salicylate, ethyl salicylate, 1-menthyl acetate, oxanone, a-irisone, methyl cinnamate, ethyl cinnamate, butyl cinnamate, ethyl butyrate, ethyl acetate, methyl anthranilate, iso-amyl acetate, iso-amyl butyrate, allyl caproate, eugenol, eucalyptol, thymol, cinnamic alcohol, octanol, octanal, decanol, decanal, phenylethyl alcohol, benzyl alcohol, a-terpineol, linalool, limonene, citral, neral, geranial, geraniol nerol, maltol, ethyl maltol, anethole, dihydroanethole, carvone, menthone, beta -damascenone, ionone, gamma -decalactone, gamma -nonalactone, y-undecalactone, and any combinations thereof.

The one or more additional component may be a coloring agent (also called “colorant”). Any suitable coloring agent known in the art may be used. For example, and without wishing to be limiting, the coloring agent may be any suitable food grade and/or non-toxic colorant or coloring agent known in the art.

The reader will readily understand that in embodiments of the present disclosure, the additional component may include a combination of any one of the above examples of additional components.

The hashish product of the present disclosure may contain one or more cannabinoid(s). The one or more cannabinoid(s) may originate from the cannabis extract, from an additional component, or both. In some embodiments, the hashish product of the present disclosure contains one or more cannabinoid(s) in an amount sufficient for the user to experience a desired effect when consuming the hashish product. In some embodiments, the hashish product of the present disclosure may include one or more cannabinoid(s), such as THC, CBD, CBN, or any combinations thereof, in similar or different amounts. In one embodiment, the hashish product of the present disclosure contains the one or more cannabinoid(s) in an amount (the “cannabinoid content”) sufficient for the user to experience a desired effect when consuming the product. For example, the hashish product may comprise from about 5 wt.% to about 90 wt.% cannabinoid, for example up to about 60 wt.%, or up to about 50 wt.%, or up to about 40 wt.%, or up to about 30 wt.%.

The hashish product of the present disclosure may contain one or more terpene(s). The one or more terpene(s) may originate from the cannabis extract, from an additional component, or both. In some embodiments, the hashish product of the present disclosure may include the one or more terpene(s) in an amount (the “terpene content”) sufficient for the user to experience a desired entourage effect when consuming the product. For example, the hashish product may comprise from about 0.5 wt.% to about 15 wt.% terpene, for example up to about 15 wt.%, or up to about 10 wt.%, or up to about 5 wt.%, or up to about 4 wt.%, or up to about 3 wt.%, or up to about 2 wt.%, or up to about 1 wt.%.

The hashish product of the present disclosure may be described by one or more of its hardness, consistency/pliability, and color.

For example, the hashish product of the present disclosure may have a color ranging from white to black; for example, and without wishing to be limiting, the hashish product may be white, light to dark yellow, light to dark brown, tan through golden or blond, reddish-brown to red, black, or any color therebetween. The color signal of the hashish product can be measured using any suitable method known in the art. In one non-limiting example, the colour signal may be determined by visual inspection and comparison to known colour charts. In another non-limiting example, the colour signal may be determined using reflectance spectrophotometer ASTM standard test methodology. Tristimulus L*, a*, b* values are measured from the viewing surface of the hashish product. These L*, a*, b* values are reported in terms of the CIE 1976 color coordinate standard. Color differences can be calculated according to method ASTM D2244-99 “Standard Test Method for Calculation of Color Differences from Instrumentally Measured Color Coordinates.” Another possible variant is to apply on the hashish product, a material that is reflective to an external source of illumination, such as UV light. This approach would make the hashish product easier to locate by a user when there is little or no ambient light; a UV light source would make the hashish product visible in the dark.

Advantageously, in some embodiments an additional color signal is applied on at least a portion of an external surface of the hashish product. For example, the color signal can be applied after production of the hashish product. Alternatively, the color signal can be applied during production of the hashish product; for example, when using an extruder, the color making up the color signal can be added into the extruder through an inlet located close to the extruder outlet / die so as to incorporate the color signal at the surface of the hashish product. Note that, in such embodiments, the color signal is not necessarily uniform over the hashish product. Applications are contemplated where the color signal is applied on only a portion of the hashish product, the remainder of the hashish product being without such color signal. It is also possible to apply to the hashish product two or more color signals.

In a specific and non-limiting example, a color signal that has been found adequate to create a contrast in white environment is one where the value L* is in the range from 0 to 50. In that range, the parameters a*, b* can take any valid value, still the color signal will create a contrast against the white environment. In a different environment such as a dark environment, the value L* could be in the range from 60 to 100 to produce a light shade that would stand out on a dark background.

In some embodiments, the hashish product of the present disclosure may have a hardness characteristic that may range from resinous to very hard; for example, and without wishing to be limiting, the hashish product may be resinous, paste-like, very soft, soft, moderately soft, moderately hard, hard, very hard, or any consistency therebetween. The pliability of the hashish product of the present disclosure may range from malleable to brittle; for example, and without wishing to be limiting, the hashish product may be very malleable, malleable, breakable, brittle, very brittle, or any level of pliability therebetween.

The hardness, consistency/pliability, color, and other characteristics of the hashish product will depend on the type of cannabis trichomes provided, the process used to obtain the cannabis extract, impurities (i.e., plant material, waxes, etc.) remaining in the hashish product, the conditions used in the production of the hashish product, and the additional components included (if any) in the hashish product. The hardness, consistency/pliability of the hashish product can be determined using any suitable method known in the art, for example but no limited to using a food texture analysis technique / equipment known in the art (e.g., Brookfield CT3 Texture Analyzer, Ametek Inc., USA).

### Methods of Using Hashish

Hashish products are typically used for recreational or medicinal purposes. For example, hashish can be used to achieve a desired effect in a user, such as a psychoactive effect, a physiological effect, or a treatment of a condition. By “psychoactive effect”, it is meant a substantial effect on mood, perception, consciousness, cognition, or behavior of a subject resulting from changes in the normal functioning of the nervous system. By “physiological effect”, it is meant an effect associated with a feeling of physical and/or emotional satisfaction. By “treatment of a condition”, it is meant the treatment or alleviation of a disease or condition by absorption of cannabinoid(s) at sufficient amounts to mediate the therapeutic effects.

The terms “treating”, “treatment” and the like are used herein to mean obtaining a desired pharmacologic and/or physiologic effect. The effect may be prophylactic, in terms of completely or partially preventing a disease, condition, or symptoms thereof, and/or may be therapeutic in terms of a partial or complete cure for a disease or condition and/or adverse effect, such as a symptom, attributable to the disease or disorder. “Treatment” as used herein covers any treatment of a disease or condition of a mammal, such as a dog, cat or human, preferably a human.

In certain embodiments, the disease or condition is selected from the group consisting of pain, anxiety, an inflammatory disorder, a neurological disorder, a psychiatric disorder, a malignancy, an immune disorder, a metabolic disorder, a nutritional deficiency, an infectious disease, a gastrointestinal disorder, and a cardiovascular disorder. Preferably the disease or condition is pain. In other embodiments, the disease or condition is associated with the feeling of physical and/or emotional satisfaction.

In the context of recreational use, the “effective amount” administered and rate and time-course of administration, will depend on the desired effect associated with a feeling of physical and/or emotional satisfaction in the subject.

In the context of health and wellness use, the “effective amount” administered and rate and time-course of administration will depend on the nature and severity of the disease or condition being treated and typically also takes into consideration the condition of the individual subject, the method of administration and the like.

### Manufacturing Process

The hashish product of the present disclosure may be produced by mixing the components thoroughly to provide a substantially homogeneous resinous mixture. For example, the mixing may be performed with mechanical mixing. By the term “mechanically mixing” or “mechanical mixing”, it is meant mixing using any suitable mechanical means. The mechanical means may be a plurality of interpenetrate helicoidal surfaces within an elongated enclosure. One non-limiting example of such a device is an extruder apparatus. An extruder apparatus may have a single extruder screw or twin extruder screws, and can be configured to have one or more mixing zones, one or more temperature zones, and one or more input zones (for introduction of material, for example isolated cannabis trichomes and/or additional components).

An extruder is a machine used to perform the extrusion process. Manufacturing by extrusion occurs when a material (usually pellets, dry powder, rubber, plastic, metal bar stock or food) is heated and pushed through a die assembly. A die is a mold that shapes the heated material as it is forced through a small opening from the inside of the extruder to the outside. Using a system of barrels or cylinders containing interpenetrate helicoidal surfaces, e.g., screw pumps or extruder screws, the extruder can mix the ingredients while heating and propelling the extrudate through the die to create the desired shape.

An extruder can have a single extruder screw or twin extruder screws, and can be configured to have one or more mixing zones, one or more temperature zones, and one or more input zones. The input zones are used for introduction of material. The mixing zones apply compression and shear forces to the input materials, blending until they are homogenized. The extruder die assembly may perform a variety of functions: it may form or shape the extrudate, it may divide the extrudate into multiple extrudates, it may inject one or more component into the extrudate, and it may compress and reduce the cross-sectional area of the extrudate.

Single screw extruders are known in the art - the screws of such extruders comprise grooves and may be cylindrical, conical, tapered and the likes as described for example in CA 2,731,515, US 6,705,752, CN101954732 and CN201792480, where each of which is herein incorporated by reference in its entirety. Twin screw extruders are also known in the art - screws of such extruders may be parallel or non-parallel, converging or non-converging, with or without differential speed, counter or non-counter rotating as described for example in US 6,609,819, WO 2020/220390, WO 2020/220495 and US 2010/0143523, where each of which is herein incorporated by reference in its entirety. Single screw and twin screw arrangements may also be integrated within a single extruder device, as described for example in US 10,124,526, which is herein incorporated by reference in its entirety. It will be readily appreciated that extruders have flexible configuration (in terms of mixing zones, temperature zones, input zones, etc.) and that any suitable configuration of the extruder apparatus that produces the hash product may be used within the context of the present disclosure.

The mechanical mixing means may be applied to the isolated cannabis trichomes under conditions sufficient to obtain a heated, cohesive, continuous, and substantially homogenous resinous mixture. The conditions or variables that can be modified during production are discussed later in this text.

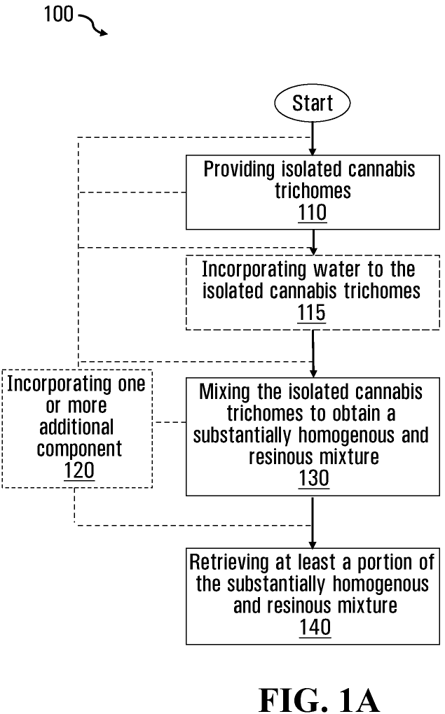

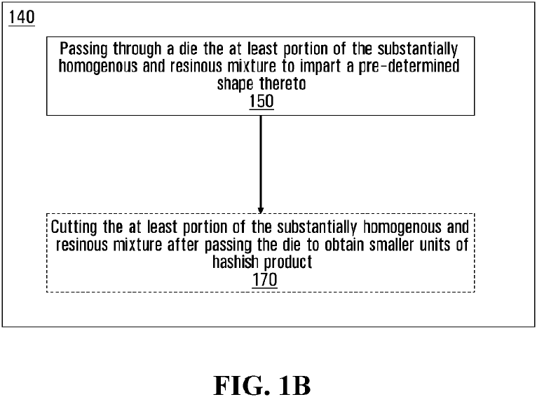

FIG. 1A shows a non-limiting example of a process 100 for producing a hashish product in accordance with an embodiment of the present disclosure. The process 100 includes providing a batch of cannabis trichomes at step 110 (alone or together with one or more additional components as will be described later in this text).

In one non-limiting example, the isolated cannabis trichomes may include trichomes isolated from a single cannabis strain. In another non-limiting example, the isolated cannabis trichomes may include trichomes isolated from a plurality of distinct cannabis plant strains, which may have different respective cannabinoid concentrations. The choice of one over the other may be driven by practical considerations, such as but not limited to inventory management consideration, the desired cannabinoid content of the hashish product, the desired dosage and/or user experience, and the like. It is known amongst consumers of hashish and other cannabis products that using isolated cannabis trichomes produced from more than one strain of cannabis plant may allow a user to tune the psychoactive and/or entourage effect obtained by consuming the product. The mixing of cannabis plant strains may also allow adjustments to the final concentration of a component of the product, for example but not limited to the cannabinoid content. Additionally, use of more than one strain allows for improved product and waste management - important in commercial production. The isolated cannabis trichomes can be kief.

The process 100 may further comprises an optional step 115 of incorporating water to the pre-treated isolated cannabis trichomes prior to the mixing step, as further described below. Water may be incorporated in the form of steam, liquid, ice, or a combination. The water incorporated may be distilled, reverse osmosis and/or microfiltered water. In some embodiments, water may be incorporated to have a total water content of about 20 wt.% or less. For example, a total water content of from about 5 wt.% to about 15 wt.% or any value therebetween, or in a range of values defined by any values therebetween. For example, a total water content of about 15 wt.% or less, about 14 wt.% or less, about 13 wt.% or less, about 12 wt.% or less, about 11 wt.% or less, about 10 wt.% or less. For example, a total water content of from about 10 wt.% to about 15 wt.%, from about 10 wt.% to about 12 wt.%.

It will be readily appreciated that the total water content of the isolated cannabis trichomes may be adjusted to any desired/target value. The relative amount of water being incorporated into the pre-treated isolated cannabis trichomes at optional step 115 may be dependent upon several factors, as further described below, such as the extrusion conditions, and/or the desired physical properties of the hashish product.

At step 130, the batch of isolated cannabis trichomes is mixed to obtain a substantially homogenous and resinous mixture. Such mixing may be performed mechanically with an extruder, for example. The pre-isolated cannabis trichomes are mixed under conditions sufficient to obtain a substantially homogenous and resinous mixture.

For example, such conditions may include shear and/or pressure, and optionally temperature, which may be varied to alter the characteristics of the hashish product. Such characteristics may include, but without being limited to, stiffness (i.e., characteristic that defines the level of malleability of the hashish product), hardness or resistance to localized deformation (i.e., characteristic that determines how easy it is to cut or separate the hashish product), toughness (i.e., characteristic that determines the likelihood that the hashish product deforms rather than fractures under an applied force), color, tactual characteristics, and the like.

For example, the pressure being applied at the mixing step 130 may be at a value of about 5 psi or more. For example, a pressure of from about 5 psi to about 1500 psi, including any ranges therein or any value therein. For example, a pressure of from about 5 psi to about 300 psi, from about 20 psi to about 300 psi, or from about 20 psi to about 250 psi, including any ranges therein or any value therein. For example, a pressure of about 20 psi, about 30 psi, about 40 psi, about 50 psi, about 100 psi, about 150 psi, about 200 psi, about 250 psi, about 300 psi. The person of skill will readily understand that a given pressure value may be obtained depending on the die and/or the mixing rotor speed that is used to form the hashish product, as described elsewhere in this text.

For example, the pressure being applied at the mixing step 130 may be performed for a time of about 0.5 minutes (30 seconds) or more. When implementing the herein described process in an elongated enclosure, such as an extruder, the pressure being applied at the mixing step 130 will be performed for a time that will vary at least based on the length of the enclosure and processing speed through the length of the enclosure. For example, the pressure being applied at the mixing step 130 may be performed for a time of from about 0.5 (30 seconds) to about 60 minutes, including any ranges therein or any value therein. For example, a time of about 5 minutes, about 10 minutes, about 15 minutes, about 20 minutes, about 30 minutes, about 40 minutes, about 50 minutes, or about 60 minutes.

For example, the temperature being applied at the mixing step 130 may be at a value of about 140° C. or less. For example, a temperature of from about 20° C. to about 120° C., including any ranges therein or any value therein. For example, a temperature of about 20° C., about 30° C., about 40° C., about 50° C., about 60° C., about 70° C., about 80° C., about 90° C., about 100° C., about 110° C., about 120° C., about 130° C., or about 140° C. In some practical implementations, the temperature at the mixing step 130 may be monitored in-process using a live temperature probe, for example.

For example, the temperature being applied at the mixing step 130 may be performed for a period of about 0.5 minutes (30 seconds) or more. When implementing the herein described process in an elongated enclosure, such as an extruder, the temperature being applied at the mixing step 130 will be performed for a time that will vary at least based on the length of the enclosure and processing speed through the length of the enclosure. For example, the temperature being applied at the mixing step 130 may be performed for a time of from about 0.5 (30 seconds) to about 60 minutes, including any ranges therein or any value therein. For example, a time of about 5 minutes, about 10 minutes, about 15 minutes, about 20 minutes, about 30 minutes, about 40 minutes, about 50 minutes, or about 60 minutes.

In one practical implementation, the mixing includes applying compression and shear forces to the isolated cannabis trichomes via a plurality of interpenetrate helicoidal surfaces within an elongated enclosure. Preferably, the elongated enclosure is an extruder device having at least one screw. The mixing shear and compressive forces can be controlled by modulating the rotational speed of at least one of the screws within the extruder. In such embodiments, the extruder screw rotation per minute (rpm) can be selected to perform the mixing step 130 at a value of for example about 5 rpm or more. For example, the extruder screw rpm can be selected in a range of from about 5 rpm to about 1000 rpm, including any ranges therein or any value therein. For example, from about 15 to about 500 rpm, or from about 25 to about 450 rpm, or from about 30 to about 400 rpm, or from about 45 to about 450 rpm including any value within any of these ranges. In such embodiment, the pressure applied by the extruder screw can be accompanied by heat to enhance mixing of the isolated cannabis trichomes, extract the resinous content of the trichomes and obtain a heated, cohesive, continuous, and substantially homogenous resinous mixture. In such embodiment, the heating and mixing can continue until a desired level of homogeneity is obtained. For example, a time of about 5 minutes, about 10 minutes, about 15 minutes, about 20 minutes, about 30 minutes, about 40 minutes, about 50 minutes, or about 60 minutes. In some embodiments, the heating and mixing continues until the desired level of homogeneity is determined by testing samples of mass retrieved from the process.

In embodiments where the heating and mixing are performed in a single screw extruder, the residence time within the extruder barrel can be directly related to the length of the barrel and the rotational speed of the single screw. To increase mixing time of the components within the barrel, the components can travel through the length of the barrel, and then be redirected to the inlet (rather than proceed through the die).

At optional step 120, one or more additional component(s) may be added at one or more steps during the process 100. For example, one or more additional component can be added to the isolated trichomes prior to, simultaneously with, or following step 110, or prior to, simultaneously with, or following the mixing step 130. Multiple additional components may be added in a single step or may be added separately in one or more consecutive steps or at different times or points along the process 100. The one or more additional components can be one or more cannabinoids, one or more terpenes, one or more flavonoids, water, one or more flavoring agents, one or more non-toxic coloring agents, or any combination thereof. The person of skill will readily appreciate that water could be added in the form of steam, liquid, ice, or in any combination thereof. When the one or more component comprises a cannabinoid, the cannabinoid may be provided in the form of a cannabis extract (including a crude extract, or a winterized extract), a distillate, an isolate, cannabis rosin, cannabis resin, cannabis wax, or cannabis shatter.

In some embodiments, the one or more additional component may be incorporated during the process to produce the hashish product and thus may be substantially homogeneously distributed throughout the hashish product. Alternatively, or additionally, the one or more additional component may be substantially homogenously distributed on at least a portion of a surface of the hashish product, for example as a coating. For example, the portion of the surface of the hashish product may include at least 20%, at least 30%, at least 40%, at least 50%, at least 60%, at least 70%, at least 80%, at least 90%, or 100% of the surface of the hashish product. By “substantially homogeneously distributed”, it is meant that the amount of the one or more additional component is uniform on the at least portion of the surface of the hashish product.

In some embodiments, the one or more cannabinoids can be in extracted and purified form and may include a crude cannabis extract, a cannabis distillate, a cannabis isolate, a winterized cannabis extract, cannabis rosin, cannabis resin, cannabis wax, or cannabis shatter, or any possible combination thereof.

In some embodiments, the one or more terpenes may include one or more terpenes which are endogenous to the cannabis strain or plurality of cannabis strains from which stem the isolated cannabis trichomes. The one or more terpenes may include one or more terpenes that are not naturally found in the one or more cannabis strain(s) from which stem the isolated cannabis trichomes.

Once the substantially homogenous and resinous mixture is obtained at step 130, at least a portion of the substantially homogenous and resinous mixture is retrieved at step 140 to obtain an individual unit of hashish product having a cohesive mass of the isolated trichomes.

The reader will understand that the process 100 may include several steps following exit of the substantially homogenous and resinous mixture from the mixing procedure. For example, the substantially homogenous and resinous mixture can be passed through a die at an optional step 150, which may be configured to impart a pre-determined shape to the resinous mixture. The size and shape imparted to the hashish product may be any desired shape, which will be determined by the size and shape of the perforations in the die. For example, and without wishing to be limiting in any manner, the hashish product may be shaped into an elongated product with a profile that is a circle; triangle; a rectangle, square, pentagon, hexagon, or any other polygonal shape; a logo; or any other more complex design. In another example, the hashish product may be formed to have a shape that elongate, curved, shell-like, or other shape similar to pasta. In yet another embodiment, the hashish product may be formed into a more functional shape, such as that of pull-apart candy or form described in US 2009/0304897 (which is herein incorporated by reference in its entirety), using a die with a plurality of openings. Alternatively, the substantially homogenous and resinous mixture can be formed simply by proceeding to a cutting step, as described below, without passing through a die.

At optional step 170, the extruded hashish product may be subjected to a transverse cutting operation to cut the extruded hashish product. The solid or semi-solid hashish product may be cut according to a pre-determined cutting pattern, a pre-determined weight, or a pre-determined length to obtain smaller units of hashish product for a pre-determined packaging size.

The shape and size of the resulting hashish product will be dependent on the shape of the die and how the product is cut. The finished hashish product may be of a size that is suitable for multiple portions of hashish (that is, a user may remove a desired portion size for each use), or may be a size suitable for a single use (that is, a ready-to-use product).

Optionally, a cooling step 160 may be performed to cool down the substantially homogenous and resinous mixture to obtain a solid or semi-solid hashish product, either prior to passing through the die, after passing through the die, prior to cutting, after cutting, or any combination thereof.

The product can then proceed to subsequent steps required for commercialization, for example the hashish product can be packaged in a step 180.

### Practical Implementation

There are several options to implement the herein described process 100.

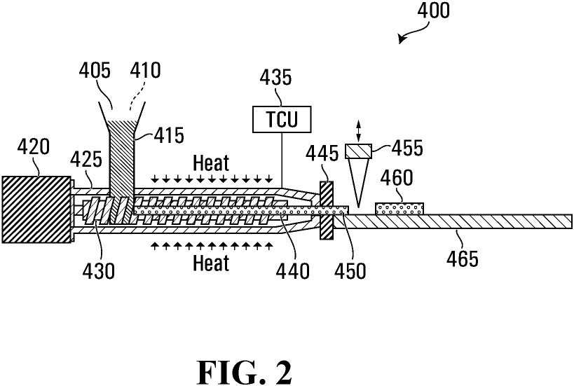

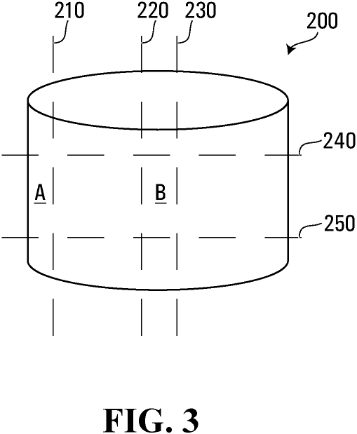

FIG. 2 illustrates a system 400 for implementing the process 100 to make individual units of hashish product 460 in accordance with an embodiment. The system 400 includes an extruder apparatus 425 that uses mechanical mixing means to amalgamate the pre-treated isolated cannabis trichomes 405 (and optionally one or more additional component(s) 410) into a coherent and substantially homogenous cohesive mass 450.

In this embodiment, the system 400 further comprises a feed hopper 415 through which the pre-treated isolated cannabis trichomes 405 (and optionally the one or more additional component(s) 410) are fed. As discussed previously, non-limiting examples of such one or more additional component(s) 410 include terpenes, flavonoids, water in the form of steam, ice or liquid, cannabinoids in the form of crude extracts, distillates, isolates, winterized cannabis extracts, rosin, shatter, or resins, or any combinations thereof. In another embodiment, at least a portion of the one or more additional component(s) 410 may be fed into the extruder apparatus 425.

The extruder apparatus 425 is powered by a motor 420 that drives at least one extruder screw 430 to apply pressure and mechanical shear on the pre-treated isolated cannabis trichomes 405 (and optionally the one or more additional component(s) 410) entering the extruder 425. For example, the extruder screw 430 may be configured for applying compression and shear forces to the pre-treated isolated cannabis trichomes 405 via a plurality of interpenetrate helicoidal surfaces present along at least a portion of the extruder screw 430.

When desired, the system 400 may also implement heating, such as within one or more predetermined portions (each a “heating zone”) of the extruder apparatus 425, or throughout the length of the extruder apparatus 425, depending on specifics applications. The operating parameters of the extruder apparatus 425, such as those discussed previously (e.g., the heating temperature and extruder screw rotation per minute (rpm)), can be selected to alter residence time of the resinous mixture 440 (or pre-treated isolated cannabis trichomes 405) in the extruder apparatus 425 to obtain the cohesive mass 450. Advantageously, it has been observed that operating parameters such as heat and extrusion speed change the pressure experienced at the die and may alter the characteristics of the hash product discussed above.

In some embodiments, the heating may additionally advantageously assist in homogeneous mixing of the pre-treated isolated cannabis trichomes 405 and optional additional components 410 to form the cohesive mass 450.

In some embodiments, the heating time may be of about 5 minutes, about 10 minutes, about 15 minutes, about 20 minutes, about 30 minutes, about 40 minutes, about 50 minutes, or about 60 minutes, depending on the specifics of an application, in each of the one or more heating zones of the extruder apparatus 425.

In some embodiments, the pressure applied by the extruder screw 430 is accompanied by heat to enhance mixing of the batch of pre-treated isolated cannabis trichomes 405 (and optionally the one or more additional component(s) 410), and/or further extract the resinous content of the pre-treated isolated cannabis trichomes and obtain a heated, cohesive, continuous and substantially homogenous resinous mixture 440.

In some embodiments, the heat may be applied through a heating element (not shown) that is embedded with the extruder screw 430 and extends along the entire or part(s) of the length of the extruder screw 430. In another embodiment, the heat may be applied through a heated jacket (not shown) that partially, or entirely, surrounds the extruder apparatus 425. To control the amount of heat input to the extruder and ensure that the quality of the resinous mixture 440 would not be compromised, a temperature controlling unit (TCU) 435 can also be associated with the extruder apparatus 425 to monitor heat within the extruder apparatus 425 and take any necessary action in the event of major deviations from the intended extrusion temperature.

For example, the temperature controlling unit (TCU) 435 may include a thermometer (not shown) that is connected to the exterior body of the extruder with its distal end in contact with the resinous mixture 440 recording an average resinous mixture temperature (T1). In another embodiment, the thermometer may be connected to the exterior body of the extruder apparatus 425 with its distal end attached to the outer surface of the extruder apparatus 425 recording an average operating temperature (T2) wherein T2= T1 -ΔT with ΔT being a temperature offset.

The resinous mixture 440 then exits the extruder apparatus in the form of an elongated, continuous solid or semi-solid cohesive mass 450. Optionally, the extrusion apparatus 425 may include a die 445 at the outlet thereof, which may impart any pre-determined shape to the cohesive mass 450. At that point in the process, the long and continuous solid or semi-solid cohesive mass 450 can be subjected to ambient temperature and pressure.

A cutting means 455 may be placed downstream of the extruder die 445. The cutting means 455 may be configured to cut the cohesive mass 450 according to a pre-established cutting pattern. In a non-limiting example of implementation, the pre-established cutting pattern may comprise cutting the cohesive mass 450 along a transverse axis and at pre-determined time intervals to obtain hashish product unit 460 of a pre-determined length and/or weight. For example, to obtain a plurality of hashish product units 460 with consistent dimensions and/or weight, the cutting means 455 can act intermittently to cut the cohesive mass 450 into individual units of hashish product 460. The individual units of hashish product 460 could be further transferred onto a flat conveyor belt 465 or fall under gravity over an inclined conveyor belt (not shown) and sent for packaging and/or storage.

### Segmentation Test

The textural crumbliness of the herein described hashish product can be assessed using a segmentation test. In this test, a hashish product is segmented along an axis using a cutting blade and the amount of residual product determined thereafter.

The test procedure is as follows:
1. 100 hashish product samples to be simultaneously tested (herein referred as “test samples”), which are all made in a single batch or individually but in a sufficiently controlled environment such as to ensure a high degree of uniformity between the samples are provided.
2. The test samples are conditioned for 1 h at a temperature of 20° C. and at a humidity level of 40%.
3. Each test sample is tested by placing same on a support surface. For test samples that are not spherical, the test samples are placed on the support surface in an orientation such that the same side of the test samples will face up, if applicable. A single blade is then used to slice the test sample along a single line to obtain substantially two identical segments.
4. Each segment is then weighted on an analytical balance, such as a Mettler Toledo™ NewClassic ME Analytical Balances (Fisher Scientific, USA), and the amount of loss material is reported for each segment as per the following ratio S_{w}/E_{w} where Sw represents the segment weight and E_{w} represents the expected weight. A ratio of 0.90 or less is considered a failure; loss of at least 10 wt.% hashish indicates a failure of the test.
5. Each test sample is classified into respective pass/fail groups based on the ratio determined for the respective pair of segments. The probability of failure per single hashish product sample failure is computed by dividing the number of hashish products that have failed by 100, which is the total number of test samples.

Note that for the purpose of the present description, the above defined test procedure will be referred to as a “segmentation test”.

According to the present disclosure, the probability of failure per hashish product does not exceed 0.25.

### Marker Distribution Test

The marker content and/or distribution into the hashish product can be assessed using a marker distribution test. In this test, a hashish product is segmented along several axes using a cutting blade to obtain peripheral and core portions and the marker content is determined thereafter. Note that for the purpose of the present description, this test procedure will be referred to as a “marker distribution test”.

The test procedure is as follows:
1. 100 hashish product samples to be simultaneously tested (herein referred as “test samples”), which are all made in a single batch or individually but in a sufficiently controlled environment such as to ensure a high degree of uniformity between the samples are provided.
2. The test samples are conditioned for 1 h at a temperature of 20° C. and at a humidity level of 40%.
3. Each test sample is tested by placing same on a support surface. For test samples that are not spherical, the test samples are placed on the support surface in an orientation such that the same side of the test samples will face up, if applicable. As illustrated in FIG. 2, a single blade is then used to slice the test sample 200 along two 2 lines 220, 230 along a longitudinal axis thereof, substantially parallel to each other. The single blade is then used to slice the test sample 200 along two 2 lines 240, 250 along a transverse axis thereof, substantially parallel to each other. The single blade is then used to slice the test sample 200 along 1 line 210 substantially parallel to lines 220, 230, and closer to the outer edge of test sample 200. The crossing of axes 220, 230 with 240, 250 produce a core portion B whereas the crossing of axes 240, 250 with 210 produces a peripheral portion A.
4. The marker content of each of the core portion B and the peripheral portion A is then determined, for example using Mettler Toledo™ Hal. Moisture Analyzer HC103 (Fisher Scientific, USA) to quantify water content, or any other suitable technique / equipment for another marker. The marker content distribution is reported for each assay as per the following ratio B/A. A ratio of 0.85 or less or 1.15 or more is considered a failure; variability of at least 15% in the marker content indicates a failure of the test.
5. Each test sample is classified into respective pass/fail groups based on the ratio determined above. The probability of failure per single hashish product sample failure is computed by dividing the number of hashish products that have failed by 100, which is the total number of test samples.

The marker content of various portions from the same test sample can be obtained as per variations of the above described procedure to determine the marker content at various location in the test sample and, thus, determine the marker content distribution in the test sample.

The distribution of the detectable marker in the hashish product is substantially homogeneous, and the marker can be detected in at least 80 vol.% of the hashish product.

The levels (or contents) of the detectable marker in the hashish product is such that the first marker content (in a core portion of the hashish product) and the second marker content (in a peripheral portion of the hashish product) are present in a ratio first marker / second marker of from 0.85 to 1.15.

According to the present disclosure, the probability of failure per hashish product does not exceed 0.25.

### Quantification of Cannabinoids

Cannabinoid content was measured with an LC Analysis using Waters Application Note, 720006509EN (Layton, C.; Aubin, A. J. (2019) UPLC Separation for the Analysis of Cannabinoid Content in Cannabis Flowers and Extracts, Application Notes, Waters, (pp 1-6) with modifications as per the following:
1. A representative 0.4 g sample of dried cannabis kief/hash was weighed into a 50 mL falcon tube using a sartorius MCA225S Cubis II Balance.
2. 25.0 ml of a 70:30 Acetonitrile-Water was added to the Falcon tube using a Eppendorf Repeater E3.
3. The mixture was vortexed for 5 mins at 2500 RPM using a VWR DVX-2500 Digital Multi-tube Vortexer.
4. The mixture was sonicated in a Branson Bransonic® M Mechanical bath 8800 for 20 mins with intermittent vortexing every 5 minutes.
5. The mixture was centrifuged at 4000 x g for 5 minutes using a Thermo Scientific Sorvall Legend™ XFR Centrifuge.
6. 9.9 mL of 70:30 Acetonitrile:Water was added to a 15 mL Falcon Tube using an Eppendorf Repeater E3.
7. 0.1 mL of Extract from (5) was added to (6).
8. The mixture was vortexed for 5 mins at 2500 RPM using a VWR DVX-2500 Digital Multi-tube Vortexer.
9. The mixture was centrifuged at 4000 x g for 5 minutes using a Thermo Scientific Sorvall Legend XFR Centrifuge.
10. 1.5 mL was transferred to a 12×32 mm HPLC screw top vial using a 3 mL Syringe with Luer-Lok with an attached 13 mm, 0.2um Acrodisc Syringe Filter.
11. The Sample was then Analyzed on a Waters UPLC® H-Class (Waters Corporation).

### Ultra-performance Liquid Chromatography (UPLC) Procedure

A 6 point curve was made from Cannabinoids Standard Mixture (Shimadzu Cat # 220-91239-22), 1 mL x 250 ug/mL with the following Cannabinoids commonly abbreviated, THC-A, THC-V, d8-THC, d9-THC, CBD, CBD-A, CBD-V, CBN, CBG, CBG-A, CBC. Calibration range: 1-100 µg/mL. Injection: 7 µL. Wavelength 228 nm @ 4.8 nm.

TABLE 1

Mobile Phase

| | |
|---|---|
| Channel | Solution |
| Eluent A | Water with 0.1% TFA (v/v) |
| Eluent B | Acetonitrile |

TABLE 2

Gradient

| Time | Eluent A | Eluent B |
|---|---|---|
| Initial | 49 | 51 |
| 4.5 | 49 | 51 |
| 8.5 | 20 | 80 |
| 9 | 5 | 95 |
| 10 | 5 | 95 |

Results were processed using the current calibration curve and reported as mg/g.

### EXAMPLES

The following examples describe some exemplary modes of making and practicing certain compositions that are described herein. These examples are for illustrative purposes only and are not meant to limit the scope of the compositions and methods described herein.

### Example 1

In this example, a hashish product comprising a cohesive mass of isolated cannabis trichomes was manufactured in a single screw extrusion device.

A batch of 270 g of dried isolated trichomes was mixed thoroughly and placed into the hopper of an ETP1 Lab Extruder (The Bonnot Company, USA). The extruder was operated at a temperature of 40° C. and a filling auger speed of 10 RPM, with increases of 5 RPM approximately every 45 seconds. The processing time for the batch was 5 minutes.

The hashish product obtained using the above methodology had uniform color throughout and was slightly pliable immediately upon extrusion. Once cooled to room temperature, the product was slightly more brittle.

### Example 2

In this example, a hashish product comprising a cohesive mass of isolated cannabis trichomes was manufactured in a double screw extrusion device.

Briefly, kief was mixed with water to obtain a 94 wt.% wet kief mixture. The wet kief was then loaded at a feed rate of about 150 g/hour with a chiller temperature of 10° C. into a Thermo Scientific™ Pharma 11 Twin-screw Extruder equipped with a 20 mm × 2 mm die and operated with the following parameters.

TABLE 3

extruder operating parameters

| Batch # | A (°C) | Z8 (°C) | Z7 (°C) | Z6 (°C) | Z5 (°C) | Z4 (°C) | Z3 (°C) | Z2 (°C) | rpm | Torque (%) | Pressure (bar) |
|---|---|---|---|---|---|---|---|---|---|---|---|
| BBE-055 | 44 | 44 | 44 | 51 | 70 | 70 | 70 | 51 | 50 | 20-22 | 80-90 |
| BBE-0552 | 44 | 44 | 44 | 57 | 80 | 80 | 80 | 56 | 50 | 20-22 | 90-100 |
| BBE-0553 | 45 | 45 | 49 | 64 | 90 | 90 | 90 | 63 | 50 | 18-20 | 80-90 |
| BBE-0553 | 45 | 45 | 49 | 64 | 100 | 100 | 100 | 63 | 50 | 18-20 | 60 |
| BBE-0553 | 51 | 49 | 59 | 72 | 100 | 100 | 100 | 70 | 200 | 18-20 | 25-30 |
| BBE-055 | 51 | 49 | 59 | 72 | 120 | 120 | 120 | 70 | 300 | 18-20 | 15-20 |

The legend for the parameters specific to the Thermo Scientific™ Pharma 11 Twin-screw Extruder is the following:
A = die (exit) temperature;
Z8 = conveying elements, Zone 8
Z7 = conveying elements, Zone 7
Z6 = mixing elements + (½)LD reverse element, Zone 6
Z5 = mixing elements, Zone 5
Z4 = conveying elements, Zone 4
Z3 = conveying elements, Zone 3
Z2 = Inlet, Zone 2

The hashish product obtained using the above methodology had uniform color throughout and was slightly pliable immediately upon extrusion.

### Example 3

In this example, a hashish product comprising a cohesive mass of isolated cannabis trichomes was manufactured in a double screw extrusion device.

Briefly, kief was mixed with water to obtain a 94 wt.% wet kief mixture. The wet kief was then loaded at a feed rate of about 300 g/hour with a chiller temperature of 10° C. into the Thermo Scientific™ Pharma 11 Twin-screw Extruder equipped with a 20 mm × 2 mm die and operated with the following parameters.

TABLE 4

extruder operating parameters

| Batch # | A (°C) | Z8 (°C) | Z7 (°C) | Z6 (°C) | Z5 (°C) | Z4 (°C) | Z3 (°C) | Z2 (°C) | rpm | Torque (%) | Pressure (bar) |
|---|---|---|---|---|---|---|---|---|---|---|---|
| BBE-058 | 70 | 70 | 85 | 140 | 140 | 140 | 80 | 40 | 300 | 12% | 21 |
| BBE-059 | 70 | 70 | 85 | 140 | 140 | 140 | 80 | 50 | 200 | 10-12 | 20-22 |
| BBE-060 | 70 | 70 | 85 | 150 | 150 | 150 | 80 | 50 | 400 | 10-12 | 18-20 |

The results obtained were as follows
BBE-058: the hashish product was brown and malleable.
BBE-059: the hashish product was brown and malleable.
BBE-060: the hashish product was brown and malleable, with no sharkskin-like structure.

### Example 4

In this example, a hashish product comprising a cohesive mass of isolated cannabis trichomes was manufactured in a double screw extrusion device.

Briefly, kief was mixed with water to obtain a 94 wt.% wet kief mixture. The wet kief was then loaded with a chiller temperature of 10° C. into a Thermo Scientific™ Pharma 11 Twin-screw Extruder equipped with a 20 mm × 4.5 mm die with a feed rate as follows: (a) a startup feed rate of about 150 g/hour and (b) a steady state run feed rate of 150 g/hour (except BBE066, where it was 200 g/hour). Steady state was determined once temperature/pressure/torque stop fluctuating after a change in parameters and the material comes out at a constant rate = feeding rate (no stop and go output). For example, one can also wait enough time that all the material inside has been fully displaced by new input (e.g., 10 minutes was normally sufficient time with the present set up to achieve steady state after a change of parameters). The extruder was operated with the following parameters.

TABLE 5

extruder operating parameters

| Batch | | A (°C) | Z8 (°C) | Z7 (°C) | Z6 (°C) | Z5 (°C) | Z4 (°C) | Z3 (°C) | Z2 (°C) | rpm | Torque (%) | Pressure (bar) |
|---|---|---|---|---|---|---|---|---|---|---|---|---|
| BBE-066 | (a) | 60 | 60 | 60 | 60 | 60 | 60 | 60 | 50 | 100 | ---- | ---- |
| | (b) | 85 | 75 | 90 | 140 | 140 | 140 | 90 | 50 | 400 | 8-10 | 0-2 |
| BBE-068 | (a) | 35 | 40 | 40 | 40 | 40 | 40 | 30 | 30 | 100 | 35-40 | 10-20 |
| | (b) | 85 | 90 | 90 | 90 | 90 | 90 | 30 | 30 | 150 | 7 | 0-1 |
| BBE-069 | (a) | 45 | 60 | 60 | 60 | 60 | 60 | 30 | 30 | 100 | 15 | 13 |
| | (b) | 95 | 130 | 130 | 130 | 130 | 130 | 30 | 30 | 100 | 7 | 0-1 |

The results obtained were as follows.
BBE-066: the hashish product had a smooth texture.
BBE-068: when the temperature at zone 8 was set at 40° C. steady state, the hashish product was malleable and when squished, gave way easily but not sticky. When the temperature at zone 8 was set at 90° C., the hashish product was slightly darker and malleable, and when squished it gave way less easily and was more resinous.
BBE-069: the hashish product was acceptable.

### Example 5

In this example, a hashish product comprising a cohesive mass of isolated cannabis trichomes was manufactured in a double screw extrusion device.

Briefly, kief was mixed with water to obtain a 94 wt.% wet kief mixture. The wet kief was then loaded with a chiller temperature of 10° C. into a Thermo Scientific™ Pharma 11 Twin-screw Extruder equipped with a 20 mm × 4.5 mm die with a feed rate as follows: (a) a startup feed rate of about 150 g/hour and (b) a steady state run feed rate of about 150 g/hour. The extruder was operated with the following parameters.

TABLE 6

extruder operating parameters

| Batch | | A (°C) | Z8 (°C) | Z7 (°C) | Z6 (°C) | Z5 (°C) | Z4 (°C) | Z3 (°C) | Z2 (°C) | rpm | Torque (%) | Pressure (bar) |
|---|---|---|---|---|---|---|---|---|---|---|---|---|
| BBE-070 | (a) | 55 | 70 | 70 | 70 | 70 | 70 | 30 | 30 | 100 | 15% | 8 |
| | (b) | 70 | 75 | 75 | 75 | 75 | 75 | 30 | 30 | 150 | 10 | 3 |
| | (b) | 90 | 100 | 110 | 110 | 110 | 110 | 30 | 30 | 150 | 10 | 3 |

The results obtained were as follows. The hashish product does not change color upon increase in temperature at zone 8. The best sticky hashish product was obtained with a temperature at zone 8 at 100 - 110° C.

### Example 6

In this example, a hashish product comprising a cohesive mass of isolated cannabis trichomes was manufactured in a double screw extrusion device.

Briefly, a kief batch was loaded with a chiller temperature of 10° C. into a Thermo Scientific™ Pharma 11 Twin-screw Extruder equipped with a 20 mm × 4.5 mm die with a feed rate of 150 g/hour. The temperature of zones 3, 4 and 5 was incrementally increased as follows: (a) the temperature was set at 70° C. and the extruder was operated for 10 minutes after the product first exists the die, then (b) the temperature was increased by 30° C., (to 100° C.) and the extruder was operated for 10 minutes, and then (c) the temperature was increased by 40° C. (to 140° C.) and the extruder was operated for 10 minutes. The extruder was operated with the following parameters.

TABLE 7

extruder operating parameters

| Batch | | A (°C) | Z8 (°C) | Z7 (°C) | Z6 (°C) | Z5 (°C) | Z4 (°C) | Z3 (°C) | Z2 (°C) | rpm | Torque (%) | Pressure (bar) |
|---|---|---|---|---|---|---|---|---|---|---|---|---|
| BBE-072 | (a) | 30 | 30 | 30 | 30 | 70 | 70 | 70 | 30 | 100 | 50% | 60 |
| | (b) | 30 | 30 | 30 | 30 | 100 | 100 | 100 | 30 | 200 | 18% | 20 |
| | (c) | 30 | 30 | 30 | 30 | 140 | 140 | 140 | 30 | 200 | 10% | 20 |

The results obtained demonstrated that the hashish product went from tough/hard (a) to softer/pliable (c).

### Example 7

In this example, a hashish product comprising a cohesive mass of isolated cannabis trichomes and an additional component in the form of a cannabinoid, was manufactured using a twin screw extruder.

In this example, the additional component was added separately from the kief, and more particularly was loaded into the extruder downstream from the kief intake. Briefly, a kief batch (having a cannabinoid concentration of 33.5 wt.%) was loaded with a chiller temperature of 10° C. into a Thermo Scientific™ Pharma 11 Twin-screw Extruder equipped with a 4.5 mm die with a feed rate of 120 g/hour. Where indicated, a cannabinoid distillate (84.4 wt.%) was also fed into the Extruder at a rate of 0.29 g/min to obtain a hashish product with a cannabinoid concentration of 40 wt.%. The startup phase had an RPM of 100 rpm and the steady state had an RPM of 200 rpm.

The extruder was operated with the following parameters.

TABLE 8

extruder operating parameters

| Batch | A (°C) | Z8 (°C) | Z7 (°C) | Z6 (°C) | Z5 (°C) | Z4 (°C) | Z3 (°C) | Z2 (°C) | Torque (%) | Pressure (bar) | Distillate |
|---|---|---|---|---|---|---|---|---|---|---|---|
| BBE-081 | 70 | 80 | 80 | 80 | 80 | 80 | 80 | 30 | 38 | 35 | No |
| | 70 | 80 | 80 | 110 | 110 | 110 | 80 | 30 | 30 | 35 | No |
| | 70 | 80 | 80 | 120 | 120 | 120 | 80 | 30 | 28 | 35 | No |
| | 70 | 80 | 80 | 120 | 120 | 120 | 80 | 30 | 22 | 20-25 | Yes |

The hashish product obtained with the addition of distillate resulted in a substantially homogeneous product with no apparent phase separation or heterogeneous portions thereof.

### Example 8

In this example, a hashish product comprising a cohesive mass of isolated cannabis trichomes and a marker, namely an additional component in the form of a cannabinoid.

In this example, the additional component was added together with the kief, and more particularly was mixed with kief to obtain a mixture and the mixture was then loaded into the extruder. Briefly, a 99.9 wt.% CBD isolate (A1) was mixed with a kief batch (A2) via mechanical mixing with a KitchenAid™ (B) or via manual mixing (C). The respective mixtures (B) or (C) were separately processed in a Thermo Scientific™ Pharma 11 Twin-screw Extruder equipped with a 20 mm × 2 mm die with a feed rate of 120 g/hour to make hashish products (D) and (E), respectively. The startup phase had an RPM of 100 rpm and the steady state had an RPM of 200 rpm.

The extruder was operated with the following parameters.

TABLE 9

extruder operating parameters

| Batch | A (°C) | Z8 (°C) | Z7 (°C) | Z6 (°C) | Z5 (°C) | Z4 (°C) | Z3 (°C) | Z2 (°C) | Torque (%) | Pressure (bar) |
|---|---|---|---|---|---|---|---|---|---|---|
| BBE-082 | 70 | 80 | 80 | 110 | 110 | 110 | 80 | 30 | 16-18 | 15-20 |

Characterization of cannabinoid content in the starting materials and in the as-produced hashish was performed with UPLC. The results are the following were the average and standard deviation (STDEV) calculations were obtained from 15 samples that were cut from a single as-produced hashish unit:

TABLE 10A

CBD content from 15 extruded samples

| | Average CBD (%) | STDEV total CBD | CBD % Variance | CBD Range |
|---|---|---|---|---|
| A1 | 99.9 | N/A | N/A | N/A |
| A2 | 0 | N/A | N/A | N/A |
| B | 11.4 | 0.36 | 3.19 | 1.5 |
| C | 11.6 | 0.57 | 4.96 | 2.1 |
| D | 11.5 | 0.54 | 4.77 | 2.2 |
| E | 11.4 | 0.23 | 2.05 | 0.9 |

TABLE 10B

THC content from 15 extruded samples

| | Average THC % | STDEV total THC | THC % Variance | THC Range | Moisture (%) |
|---|---|---|---|---|---|
| A1 | N/A | N/A | N/A | N/A | N/A |
| A2 | 27.2 | 0.60 | 2.22 | 2.4 | 6.15 |
| B | 23.6 | 0.53 | 2.25 | 2.2 | N/A |
| C | 23.9 | 0.83 | 3.48 | 2.4 | N/A |
| D | 23.4 | 0.54 | 2.32 | 1.8 | 3.80 |
| E | 22.8 | 0.50 | 2.20 | 2.0 | 3.59 |

As is known in the art, standard deviation measures the amount of variability among the numbers in a data set. It calculates the typical distance of a data point from the mean of the data. If the standard deviation is relatively large, it means the data is quite spread out away from the mean. If the standard deviation is relatively small, it means the data is concentrated near the mean. Variance is the expectation of the squared deviation of a random variable from its mean. Variance is a measure of dispersion, meaning it is a measure of how far a set of numbers is spread out from their average value.

### Example 9

In this example, a hashish product comprising a cohesive mass of isolated cannabis trichomes and a marker, namely an additional component in the form of a cannabinoid mixture.

In this example, the additional component was added separately from the kief, and more particularly was loaded into the extruder downstream from the kief intake. Briefly, a first hashish product (B) was made by loading a kief batch (A2) into a Thermo Scientific™ Pharma 11 Twin-screw Extruder equipped with a 20 mm × 2 mm die with a feed rate of 120 g/hour. A second hashish product (C) was made by loading a kief batch (A2) and a 78.57 wt.% CBD and 2.85 wt.% THC distillate (A1) in the extruder. The kief feed rate was 120 g/hour and the distillate feed rate was 0.29 g/min (the distillate was fed through intake valve at zone 4 whereas the kief was fed through intake zone 2). The startup phase had an RPM of 100 rpm and the steady state had an RPM of 200 rpm.

The extruder was operated with the following parameters.

TABLE 11

extruder operating parameters

| Batch | A (°C) | Z8 (°C) | Z7 (°C) | Z6 (°C) | Z5 (°C) | Z4 (°C) | Z3 (°C) | Z2 (°C) | Torque (%) | Pressure (bar) |
|---|---|---|---|---|---|---|---|---|---|---|
| BBE-081 (B) | 70 | 80 | 80 | 120 | 120 | 120 | 80 | 30 | 28 | 35 |
| BBE-081 (C) | 70 | 80 | 80 | 120 | 120 | 120 | 80 | 30 | 22 | 20-25 |

Characterization of cannabinoid content in the starting materials and in the as-produced hashish was performed with UPLC. The results are the following were the average and standard deviation (STDEV) calculations were obtained from 15 samples that were cut from a single as-produced hashish unit:

TABLE 12A

CBD content from extruded 15 samples

| | Average CBD (%) | STDEV total CBD | CBD % Variance | CBD Range |
|---|---|---|---|---|
| A1 | 78.57 | N/A | N/A | N/A |
| A2 | 0 | N/A | N/A | N/A |
| B | 0.0 | N/A | N/A | N/A |
| C | 5.4 | 0.35 | 6.39 | 1.0 |

TABLE 12B

THC content from 15 extruded samples

| | Average THC % | STDEV total THC | THC % Variance | THC Range | Moisture (%) |
|---|---|---|---|---|---|
| A1 | 2.85 | N/A | N/A | N/A | N/A |
| A2 | 27.2 | 0.60 | 2.22 | 2.4 | 6.15 |
| B | 26.8 | 0.88 | 3.27 | 3.1 | 4.95 |
| C | 25.1 | 0.74 | 2.96 | 2.6 | 3.58 |

The results obtained demonstrate that the extruder affords tight variance of the marker - i.e., here addition of one or more component, namely one or more cannabinoids. Since variance is a measure of how spread out a data set is, the results indicate an increase in homogeneity of the marker in the hashish product.

The results also show that the hashish product retains a substantial homogeneity in terms of distribution of the added one or more component, whether the one or more component is incorporated into the process prior to mixing in the extruder (example 8) or during the mixing in the extruder (example 9).

Further, the inventors have further observed that when the one or more component was incorporated into the process prior to mixing in the extruder, this mixture contained white flecks of the CBD isolate. In other words, to the naked eye, you would still be able to see and distinguish the separate components of the mixture, which suggests that with time, the CBD isolate would be prone to sedimentation, separation, or segregation, thus resulting with progressive heterogeneity. More particularly, the present inventors observed that after processing in the extruder, such white flecks of isolate were no longer present, thus suggesting that the extrusion caused the isolate and the kief to form a substantially homogeneous cohesive mass that was not obtainable with the mixing prior to the extrusion. Without being bound by any theory, the present inventors believe that the extrusion process described herein melts the CBD isolate thus spreading same more evenly within the mass of the hashish product, thus resulting with a fleckless cohesive mass.

### Comparative Example 1 - Pressed Hashish

In this example, prior art hashish products were manufactured by pressing kief according to the pressing procedure set forth in PCT/CA2020/051733 to obtain hashish bricks of substantially similar size. The cannabinoid content of 15 distinct hashish bricks was measured with HPLC and the results are presented in the following table 13.

TABLE 13

THC content from 15 pressed units

| Product # | THC Content (wt.%) |
|---|---|
| 1 | 25.11 |
| 2 | 24.01 |
| 3 | 23.63 |
| 4 | 24.18 |
| 5 | 24.57 |
| 6 | 25.11 |
| 7 | 24.27 |
| 8 | 24.95 |
| 9 | 25.47 |
| 10 | 25.14 |
| 11 | 24.75 |
| 12 | 25.33 |
| Standard deviation | 0.59 |
| Average | 24.73 |
| Coefficient of variance | 0.02 |

The results show that pressing isolated trichomes results in hashish products units having some variations in THC content between units.

### Example 10

In this example, hashish products comprising a cohesive mass of isolated cannabis trichomes were manufactured using a single screw extruder.

Extrusion was done with a 150 g kief batch (from identical cannabis strain as in comparative example 1) in an ETPI Lab extruder (The Bonnot Company, USA) with a barrel temperature of 60° C. and a rotor speed of 15 rpm with a 20 mm × 5 mm die.

The cannabinoid content of 15 distinct extruded hashish units was measured with HPLC and the results are presented in the following table 14.

TABLE 14

THC content from 15 extruded units

| Product # | THC Content (wt.%) |
|---|---|
| 1 | 28.10 |
| 2 | 27.48 |
| 3 | 27.49 |
| 4 | 28.12 |
| 5 | 28.41 |
| 6 | 28.35 |
| 7 | 27.66 |
| 8 | 27.68 |
| 9 | 28.35 |
| 10 | 27.57 |
| 11 | 27.98 |
| 12 | 28.16 |
| Standard deviation | 0.32 |
| Average | 27.96 |
| Coefficient of variance | 0.01 |

The results show that extruding isolated cannabis trichomes results in hashish products units having improved homogeneity in terms of THC content between units relatively to the THC content obtained with the pressing procedure of comparative Example 1. Again, the results obtained demonstrate that the extruder affords tight variance of the marker - i.e., here a cannabinoid. Since variance is a measure of how spread out a data set is, the results indicate an increase in homogeneity of the marker in a batch of hashish products.

Other examples of implementations will become apparent to the reader in view of the teachings of the present description and as such, will not be further described here.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

Unless otherwise defined, all technical and scientific terms used herein have the same meaning as commonly understood by a person of ordinary skill in the art to which the present invention pertains. In the case of conflict, the present document, including definitions will prevail.

All references cited throughout the specification are hereby incorporated by reference in their entirety for all purposes.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any theory or scheme of action.

Although various embodiments of the disclosure have been described and illustrated, it will be apparent to those skilled in the art considering the present description that numerous modifications and variations can be made. The scope of the invention is defined more particularly in the appended claims.

1. A hashish product, comprising a cohesive mass of isolated cannabis trichomes and a detectable marker, wherein the marker is substantially homogeneously distributed throughout the hashish product.

2. The hashish product of claim 1, wherein a first level of the marker in a discreet portion of the product is within 15% of a second level of the marker, and wherein the second level is an average level of the marker in the hashish product or in a batch of hashish products.

3. The hashish product of claim 2, wherein the first level of the marker is within 10%, or within 5% of the second level of the marker.

4. (canceled)

5. A hashish product, comprising a cohesive mass of isolated cannabis trichomes and a detectable marker, wherein the hashish product includes a first content of the marker in a core portion thereof and a second content of the marker in a peripheral portion thereof, wherein the first content and the second content are present in a ratio first content / second content of from 0.85 to 1.15.

6. The hashish product of claim 5, wherein the ratio first / second contents is of at least 0.85, at least 0.90, at least 0.95, 1.00, 1.05 or less, 1.10 or less, or 1.15 or less.

7. The hashish product of claim 1, wherein said marker includes an endogenous component of the cannabis trichomes.

8. The hashish product of claim 1, wherein said marker includes an exogenous component to the cannabis trichomes.

9. The hashish product of claim 1, wherein the marker is a cannabinoid, a terpene, a flavonoid, chlorophyll, water, or any combination thereof.

10. The hashish product according to claim 1, wherein the isolated cannabis trichomes are kief.

11. The hashish product according to claim 1, wherein the cannabis trichomes are from one or more strain(s) of cannabis plant.

12-18. (canceled)

19. A batch of hashish products, each hashish product in the batch of hashish products comprising a cohesive mass of isolated cannabis trichomes and a detectable marker, wherein the detectable marker in a discreet portion of a first hashish product is a first level of the marker, wherein the first level of the marker is within 15% of a second level of the marker, and wherein the second level is an average level of the marker in the batch of hashish products.

20. The batch of hashish products of claim 19, wherein the first level of the marker is within 10%, or within 5% of the second level of the marker.

21. The batch of hashish products of claim 19, wherein said marker includes an endogenous component of the cannabis trichomes.

22. The batch of hashish products of claim 19, wherein said marker includes an exogenous component to the cannabis trichomes.

23. The batch of hashish products of claim 19, wherein the marker is a cannabinoid, a terpene, a flavonoid, chlorophyll, water, or any combination thereof.

24-30. (canceled)

31. The batch of hashish products according to claim 19, wherein each of the hashish products in the batch comprises a cannabinoid content of from about 5 wt.% to about 90 wt.%.

32. The batch of hashish products according to claim 31, wherein the cannabinoid content is up to about 60 wt.%, or up to about 50 wt.%, or up to about 40 wt.%, or up to about 30 wt.%.

33. The hashish product according to claim 1, wherein the cohesive mass is a fleckless cohesive mass.

34. The batch of hashish products according to claim 19, wherein the cohesive mass is a fleckless cohesive mass.
